(19) United States
(12) Patent Application Publication
(10) Pub. No.: US 2010/0324142 A1
(43) Pub. Date: Dec. 23, 2010

(54) HYDROCORTISONE DERIVATIVES AND USE THEREOF FOR THERAPEUTIC OR COSMETIC INDICATIONS

(76) Inventor: John Alexander Troostwyk, London (GB)

Correspondence Address: Steinfl & Bruno, 301 N Lake Ave Ste 810, Pasadena, CA, 91101, US

(21) Appl. No.: 12/681,441

(22) PCT Filed: Sep. 30, 2008
(86) PCT No.: PCT/IB2008/002562

§ 371 Date: Jul. 16, 2010

(30) Foreign Application Priority Data
Oct. 4, 2007 (IT) MI2007A001905

(51) Int. Cl. A61K 8/365; C07C 59/353; C07C 51/34; A61Q 7/00

(52) U.S. Cl. 514/573; 562/499

(57) ABSTRACT

The present invention relates to hydrocortisone derivatives, the method of production thereof and the therapeutic or cosmetic use thereof to combat hair loss or in the treatment of cutaneous physiopathologies.

The present invention relates to hydrocortisone derivatives, the method of production thereof and the therapeutic or cosmetic use thereof to combat hair loss or in the treatment of cutaneous physiopathologies.

Excessive hair loss is a very widespread condition among the world population and may regard both sexes, though it particularly affects the male population. In the early stages, this condition leads to thinning and subsequently to a complete loss of hair, i.e. absence of hair in regions of the skin normally characterised by the presence thereof, a condition commonly referred to as alopecia.

In some cases, hair loss may be consequent to a pathological state, such as seborrhoeic dermatitis.

In other cases, hair loss can be considered not as a symptom of a pathology but simply as the aesthetically undesirable consequence of a physiological state without pathologic effects. This is the case of androgenic alopecia.

Other forms of alopecia include those of a psychogenic or stress-related type (telogen effluvium), alopecia greata and alopecia caused by hair fracture (pseudo-alopecia). Among the cicatricial forms, the most common are: lichen planopilaris; lupus; and pseudopelade of Brocq.

In yet other cases, hair loss can be caused by external factors such as, for example, irritant agents that come into contact with the scalp, or may be of an iatrogenic type, i.e. caused by the administration of drugs, for example antitumoral agents.

There is hence a greatly felt need for treatments that are able to reduce and/or arrest hair loss irrespective of the cause determining it, and which are likewise capable of inducing the regrowth of hair itself without causing undesired side effects.

The Applicant has surprisingly found that hydrocortisone derivatives as defined hereinbelow are able to arrest or in any case reduce hair loss, both that caused by generic physiopathologic states and that of an iatrogenic type, deriving above all from the use of chemotherapeutics.

The Applicant has moreover found that such hydrocortisone derivatives, when applied on skin, are able to perform an effective anti-seborrhoeic and descaling action, so that they can be used in cosmetic preparations for treatments of a skin trophic type.

Therefore, according to a first aspect, the present invention regards a compound having the formula (I):

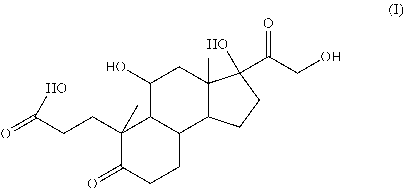

or a salt thereof.

In particular, the compound of formula (I) may be in the form of a salt wherein the counterion is selected from: sodium succinate, ammonium, metal ions, preferably of alkali or alkaline earth metals.

According to another aspect, the present invention relates to a compound of formula (I) for use as a medicament.

According to a further aspect, the present invention relates to a compound of formula (I) for use in the treatment of hair loss.

According to a further aspect, the present invention relates to a compound of formula (I) for use in the treatment of alopecia.

According to a further aspect, the present invention relates to a compound of formula (I) for use in the treatment of iatrogenic-based hair loss. According to a further aspect, the present invention relates to the use of a compound of formula (I) for the manufacture of a medicament for the treatment of any of the above-specified pathologies.

According to a further aspect, the present invention relates to the use of a compound of formula (I) in the cosmetic treatment of the skin, in particular in anti-seborrhoeic and/or descaling treatments.

According to a further aspect, the present invention relates to a pharmaceutical composition comprising a compound of formula (I).

According to a further aspect, the present invention relates to a cosmetic composition comprising a compound of formula (I).

According to a further aspect, the present invention relates to a process for producing a compound of formula (I), which comprises submitting a hydrocortisone to ozonolysis.

In a preferred embodiment, the compound according to the invention is present in a pharmaceutical or cosmetic composition.

Such composition may comprise, in addition to the compound of formula (I), at least one other ingredient selected, for example, from among: active ingredients endowed with a complementary and/or supplemental action; ingredients suitable for facilitating the transdermal delivery thereof; cosmetological excipients; aromatising agents; fragrances; colourants; stabilisers; water, in the quantity necessary to reach the desired consistency of the formulation.

Said active ingredients are preferably selected from: amino acids, such as lysine, leucine, proline (specific for the production of collagen); hyaluronic acid (essential component of the dermis), citric acid, alpha lipoic acid, glycyrrhetic acids; vitamins, such as vitamin A, vitamin E, vitamin C; ceramides; olive oil; non-saponifiables of vegetable oils; soy lecithin; shea butter; glucans; zinc oxides; natural plant extracts, such as alpha bisabolol, aloe, black currant oil, ginkgo biloba, escin, hamamelis.

Preferably, the composition of the invention further comprises at least one active ingredient selected from among: lysine, leucine, proline, non-saponifiables, hyaluronic acid, alpha lipoic acid, vitamins A, E, C, ceramides 1, 3, 6, shea butter, aloe, escin, 18-β-glycyrrhetic acid, zinc oxides, alpha bisabolol, β-glucan, glucosaminoglycans, benzyl benzoate, benzalkonium chloride, or mixtures thereof.

The quantity of the active ingredient(s) may vary according to the type of topical formulation and the planned applicative use.

Preferably, the cosmetic and/or pharmaceutical composition according to the present invention is prepared in the form of a solution or creamy and/or fluid emulsion (cream, fluid cream, gel cream), lotion, serum or gel, for topical application on the skin.

According to a preferred embodiment, the cosmetic and/or pharmaceutical composition according to the present invention is in the form of an aqueous solution.

The compound of formula (I) according to the invention can be produced according to a process that comprises at least a phase of ozonolysis of hydrocortisone.

The ozonolysis reaction can be carried out according to techniques known in the art. It is generally achieved by bubbling oxygen containing ozone through an aqueous solution of hydrocortisone. The solvent may be, for example, a hydroalcoholic mixture, in particular water/methanol in a 40/60 ratio. The aqueous solution may optionally contain hydrogen peroxide.

The ozonolysis process breaks the A ring in the position of carbon 3, producing a CO₂ molecule, thus forming the compound according to the invention as shown here below:

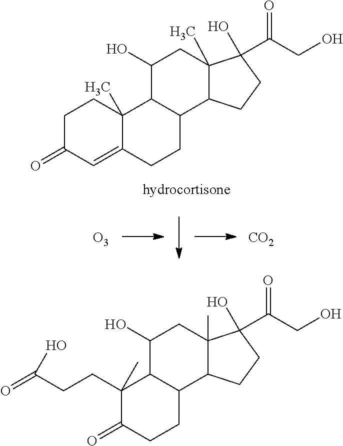

The present invention will now be further illustrated with some working examples, which are provided solely for illustrative purposes and in no way limit the scope of the invention, with reference to the following figures appended hereto:

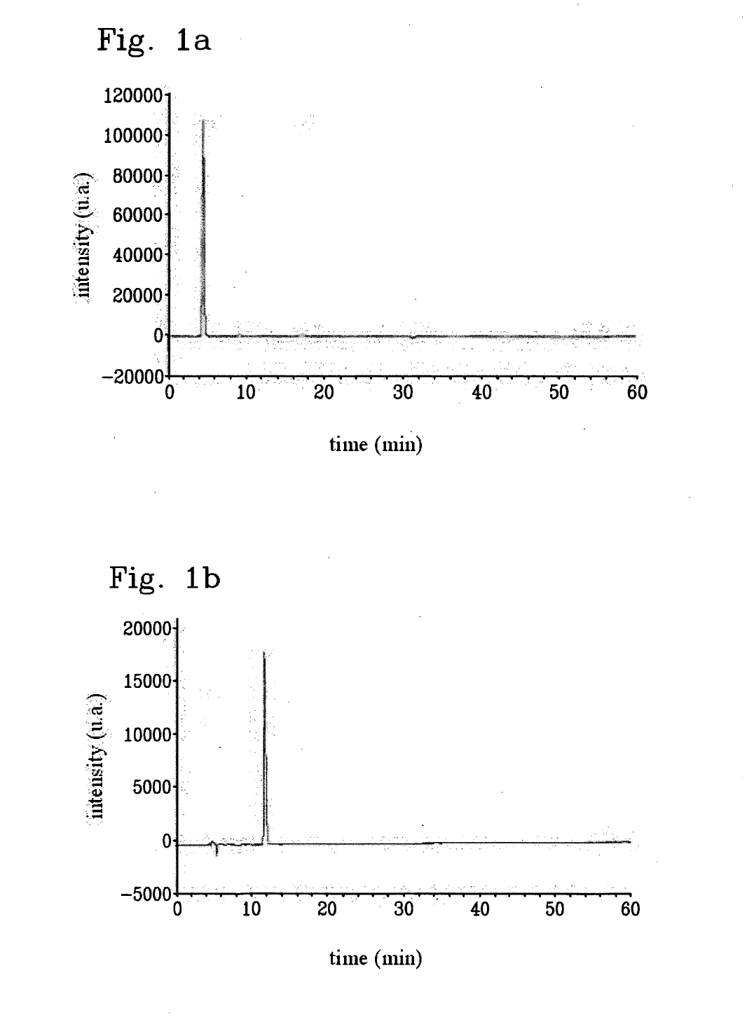

FIG. 1 shows the elution chromatogram of a compound according to the invention (FIG. 1(a)) and of hydrocortisone (FIG. 1(b));

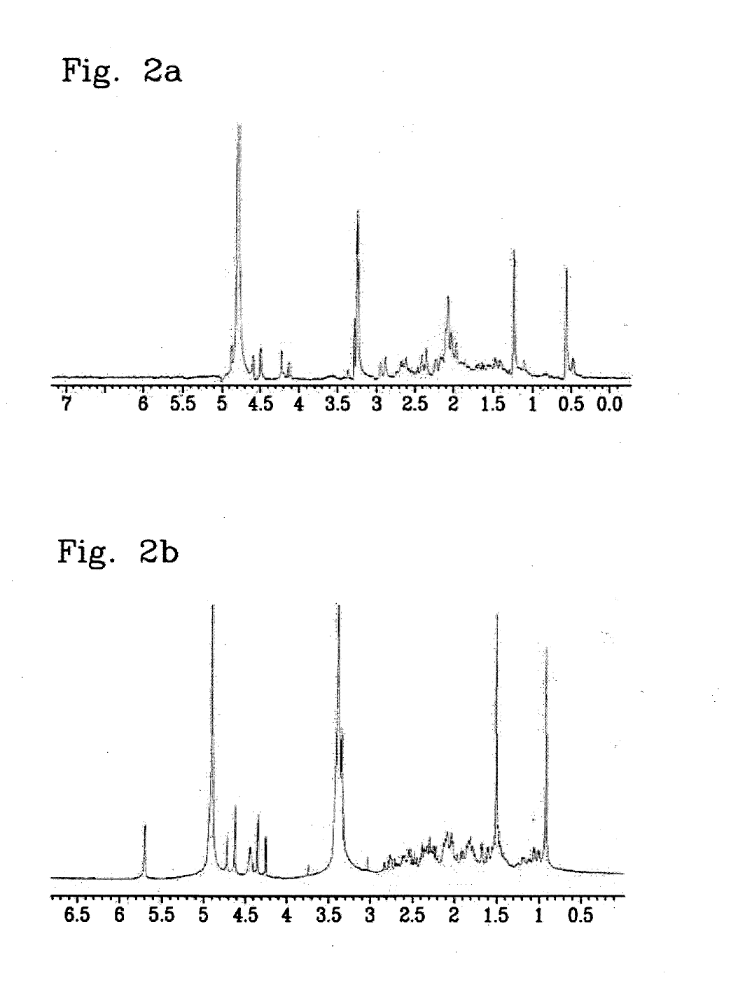

FIG. 2 shows the ¹H-NMR spectrum in deuterated methanol of a compound of formula (I) (FIG. 2(a)) and of hydrocortisone (FIG. 2(b)).

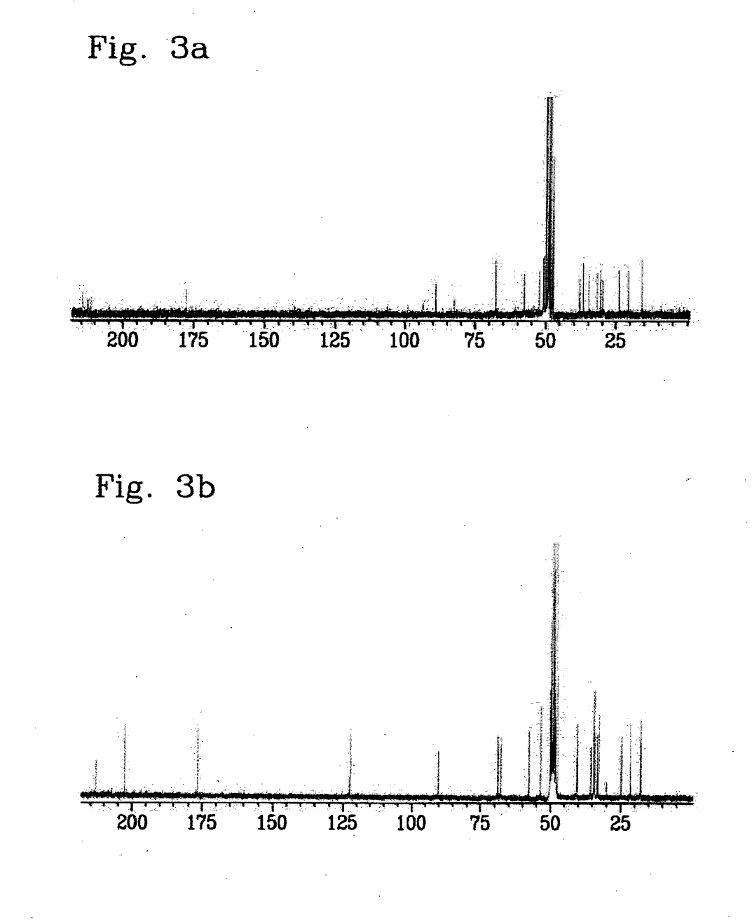

FIG. 3 shows the ¹³C-NMR spectrum in deuterated methanol of a compound of formula (I) (FIG. 3(a)) and of hydrocortisone (FIG. 3(b)).

### EXAMPLE 1

### Preparation of a Compound According to the Invention

100 mg of hydrocortisone (Fluka, HPLC grade, purity >97%) was dissolved in 20 ml of a hydroalcoholic solution of water and methanol (in a 40/60 ratio). A gaseous oxygen mixture containing 4% ozone was bubbled through the solution for approximately 20 minutes at a pressure of 1.4 bar. At the end of the treatment with ozone, the hydroalcoholic solution was left to rest for around 72 hours.

At the end of this period the pH of the solution was measured and compared with the value obtained for the same solution prior to ozonisation. The pH of the hydrocortisone solution was equal to 6.1, whereas after ozonisation the pH was equal to 2.9.

To characterize the product, the product of the ozonolysis of hydrocortisone was analysed by liquid chromatography (HPLC). A water/methanol mixture in a proportion of 40:60 was used as the eluent, in the following working conditions: eluent flow rate equal to 0.6 ml/min, time of analysis 60 minutes, working temperature of the column equal to 35° C., setting of the UV detector at wavelengths of 254 and 300 nm.

The chromatogram was recorded using a Jasco PU-2089 Plus chromatograph equipped with a Gemini C18 5 μm 110 A (Phenomenex) 4.6 mm×250 mm column, a Jasco RI-2031 Plus detector (refraction index) and a Jasco UV-2077 Plus detector (UV spectrometer).

The resulting chromatogram is given in FIG. 1(a) and shows a single well-resolved signal characterised by a different retention time compared to the chromatogram of pure hydrocortisone, which is instead shown in FIG. 1(b) and was obtained in the same experimental conditions. The complete disappearance of the signal at 12 minutes in FIG. 1(a) shows the complete absence of hydrocortisone.

The reaction product was converted into dry form by liophylisation of the hydroalcoholic solution in which it had been dissolved, and a white power was obtained. This powder was dissolved in deuterated methanol (CD₃OD) and the solution thus obtained was submitted to ¹H-NMR and ¹³C-NMR spectrometric analysis. The instrument used was an NMR Varian-Gemini 200 MHz spectrometer.

The resulting ¹H-NMR spectrum is shown in FIG. 2(a). FIG. 2(b) shows the ¹H-NMR spectrum of the hydrocortisone before ozonolysis, when it was submitted to spectrometric analysis in the same experimental conditions. In FIG. 2(a) disappearance of the singlet present in the spectrum of FIG. 2(b) is observed, due to the hydrogen bonded to the double C═C bond of the hydrocortisone. It should also be noted that the structured signal present at 4.4 ppm in FIG. 2(b) is absent in FIG. 2(a), even though it may simply have shifted to 5.0 ppm, partly covered by the intense signal of the solvent. The ¹³C-NMR spectrum of the compound obtained from the ozonolysis reaction is shown in FIG. 3(a), whereas FIG. 3(b) shows the ¹³C-NMR spectrum of hydrocortisone before ozonolysis.

Disappearance of the three signals present in FIG. 3(b) at 202, 176 and 122 ppm is observed, which may be attributed to the carbonyl carbon atom and the two adjacent carbon atoms bonded by the double C═C bond present in hydrocortisone.

In FIG. 3(a) new signals are visible at 215, 212 and 178 ppm. Such signals are compatible with the presence of carbonyl groups (the first two) and of a carboxylic acid (the third one).

In conclusion, the chromatographic analysis demonstrated that hydrocortisone completely reacted in the course of ozonization. The and ¹³C-NMR spectrometric analyses show that the olefinic double bond and the carbonyl group conjugated to it disappear, giving rise to a new carbonyl group and a carboxylic acid. The other functional groups present on the hydrocortisone molecule remain unchanged.

These analyses confirm the obtainment of the compound of formula (I), whose IUPAC name is the following:
3-[3,5-dihydroxy-3-(2-hydroxy-acetyl)-3a,6-dimethyl-7-oxo-dodecahydro-cyclo-penta[α]naphtalen-6-yl]-propionic acid

### EXAMPLE 2

### Clinical Trial (Double Blind) on Two Hundred Patients Suffering from Alopecia

The action of the compound according to the invention, produced as described in Example 1, was assessed on a sample population of two hundred people, half of whom were of the female sex. All the persons included in the sample had been affected for at least four months by physiopathological hair loss of unknown cause.

A double blind trial was set up, in which one hundred patients were administered with a solution containing the compound according to the invention in bidistilled water and one hundred received a placebo solution. The solution according to the invention contained as excipients: sodium hydroxide, benzyl benzoate and benzalkonium chloride.

At the end of the trial, it was found that in 94% of the subjects treated with the compound according to the invention hair loss was arrested within the first week of treatment. Furthermore, good hair regrowth was observed in 38% of the patients.

No substantial difference was observed between the response of the two sexes. It is worth pointing out the total absence of adverse side effects even after many months of treatment.

1. A compound having formula (I): 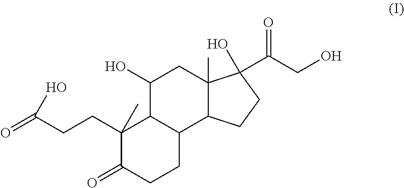
or a salt thereof.

2. The compound according to claim 1, said compound being in form of a salt whose counterion is selected from: sodium succinate, ammonium, metal ions.

3-7. (canceled)

8. A pharmaceutical composition comprising a compound according to claim 1.

9. A cosmetic composition comprising a compound according to claim 1.

10. The pharmaceutical composition according to claim 8, said composition being in the form of an aqueous solution.

11. A process for producing a compound according to claim 1, which comprises submitting a hydrocortisone to ozonolysis.

12. The process according to claim 11, wherein ozonolysis is achieved by bubbling oxygen containing ozone through an aqueous solution of hydrocortisone.

13. The process according to claim 12, wherein the aqueous solution of hydrocortisone contains hydrogen peroxide.

14. The compound according to claim 2, wherein the counterion is selected from alkali or alkaline earth metals.

15. A medicament comprising the compound according to claim 1.

16. The medicament of claim 15, wherein said compound in form of a salt whose counterion is selected from: sodium succinate, ammonium, metal ions.

17. The medicament of claim 16, wherein the counterion is selected from alkali or alkaline earth metals.

18. The medicament of claim 15, wherein the compound is comprised in an effective amount for treating hair loss.

19. The medicament of claim 15, wherein the compound is comprised in an effective amount for treating alopecia.

20. The medicament of claim 15, wherein the compound is comprised in an effective amount for treating iatrogenic-based hair loss.

21. A method for treating hair loss in an individual, the method comprising:
administering to the individual an effective amount of the compound of claim 1, wherein the administered effective amount is effective to treat hair loss in the individual.

22. A method for treating alopecia in an individual, the method comprising:
administering to the individual an effective amount of the compound of claim 1, wherein the administered effective amount is effective to treat alopecia in the individual.

23. A method for treating iatrogenic-based hair loss in an individual, the method comprising:
administering to the individual an effective amount of the compound of claim 1, wherein the administered effective amount is effective to treat iatrogenic-based hair loss in the individual.

24. A method for cosmetic treatment of an individual, the method comprising administering to the individual an effective amount of the compound of claim 1, wherein the administered effective amount is effective in cosmetic treatment of skin of the individual.

25. The method of claim 24, wherein the cosmetic treatment is an anti-seborrhoeic treatment and/or a descaling treatment.
